(19) United States
(12) Patent Application Publication
(10) Pub. No.: US 2023/0190074 A1
(43) Pub. Date: Jun. 22, 2023

(54) SHOE CARE APPARATUS AND METHOD FOR CONTROLLING THEREOF

(71) Applicant: SAMSUNG ELECTRONICS CO., LTD., Suwon-si (KR)

(72) Inventors: Heemoon JEONG, Suwon-si (KR); Minsoo KIM, Suwon-si (KR); Joongwon NA, Suwon-si (KR); Judai KIM, Suwon-si (KR); Kookjeong SEO, Suwon-si (KR)

(73) Assignee: SAMSUNG ELECTRONICS CO., LTD., Suwon-si (KR)

(21) Appl. No.: 18/113,435

(22) Filed: Feb. 23, 2023

Related U.S. Application Data
(63) Continuation of application No. PCT/KR2021/008234, filed on Jun. 30, 2021.

(30) Foreign Application Priority Data
Aug. 24, 2020 (KR) 10-2020-0106151

(51) Int. Cl. A47L 23/20; F25B 49/02; A47B 61/04; A61L 9/20; A61L 9/12; A61L 9/04; A61L 9/14

(52) U.S. Cl. CPC A47L 23/20; F25B 49/022; A47B 61/04; A61L 9/20; A61L 9/122; A61L 9/046; A61L 9/14; F25B 2600/0253; F25B 2700/151; F25B 2700/2104; F25B 2700/21172; A61L 2209/111; A61L 2209/212; A61L 2209/13

(57) ABSTRACT

A shoe care apparatus including a chamber including an air inlet and an air outlet; a first duct connected to the air outlet and having an evaporator and a condenser arranged inside; a second duct connected to the first duct and the air inlet; a holder arranged in the chamber and connected to the air inlet; a fan configured to circulate air through the first duct, the second duct, the holder, the chamber; a compressor configured to discharge a refrigerant to the condenser; a first temperature sensor configured to measure a first temperature of air heated by the condenser; and a controller configured to determine a target temperature of the heated air based on the user input, operate the compressor at the operation frequency based on the target temperature and outside air temperature, and control the operation frequency of the compressor based on the target temperature and the first temperature.

### CROSS-REFERENCE TO RELATED APPLICATION

This application is a continuation application, under 35 U.S.C. § 111(a), of International Application No. PCT/KR2021/008234, filed on Jun. 30, 2021, which is based on and claims priority under 35 U.S.C. § 119 to Korean Patent Application No. 10-2020-0106151, filed on Aug. 24, 2020 in the Korean Intellectual Property Office, the disclosure of which is incorporated by reference herein in its entirety.

### BACKGROUND

### Field

The disclosure relates to a shoe care apparatus capable of keeping the air supplied into a chamber at a constant temperature.

### Description of the Related Art

The traditional dryer or the traditional clothing care device uses a venting method for heating air brought in from outside with a heater or a closed circulation method for heating air in the chamber by continuous circulation of the air using a heat pump cycle, to supply hot air into the chamber.

The venting method, however, may cause a problem of releasing hot and humid air with bad smell to the outside and a problem of having a large temperature difference in the chamber due to the air brought in from outside.

In the case of the closed circulation method, the air is heated while constantly circulating in the device, causing continuous heat buildup and making it difficult to keep the air supplied into the chamber at a constant temperature. When the heat buildup is continued in the chamber, it may cause thermal damage to an object to be dried.

### SUMMARY

According to an embodiment, a shoe care apparatus includes a main body, a chamber formed in the main body, the chamber including an air inlet and an air outlet; a first duct connected to the air outlet and having an evaporator and a condenser arranged inside; a second duct connected to the first duct and the air inlet; a holder arranged in the chamber to hold shoes to e cleaned, the holder connected to the air inlet; a fan configured to circulate air through the first duct, the second duct, the holder, the chamber, and return to the first duct; a compressor configured to discharge a refrigerant to the condenser; a first temperature sensor configured to measure a first temperature of air heated by the condenser; a control panel configured to obtain a user input; and a controller configured to determine a target temperature of the heated air based on the user input, determine an operation frequency, operate the compressor at the operation frequency based on the target temperature and an outside air temperature, and control the operation frequency of the compressor based on the target temperature and the first temperature.

The controller may further configured to increase or decrease the operation frequency of the compressor for the first temperature to reach the target temperature in response to the first temperature reaching a preset first threshold temperature.

The controller may further configured to determine a temperature difference between the target temperature and the first temperature, determine a value of variation in the temperature difference at preset intervals, and determine a control value of the operation frequency corresponding to the temperature difference and the value of variation in the temperature difference by referring to a fuzzy table.

The controller may further configured to stop an operation of the compressor in response to the first temperature reaching a preset second threshold temperature and the operation frequency reaching a preset minimum frequency, and operate the compressor which has been stopped in response to the first temperature reduced below a preset third threshold temperature.

The shoe care apparatus may further include a third temperature sensor arranged on an inlet side of the evaporator and configured to measure temperature of the refrigerant, and the controller is further configured to stop an operation of the compressor in response to the temperature of the refrigerant equal to or higher than a preset protection temperature, and operate the compressor which has been stopped in response to the first temperature reduced below a preset third threshold temperature.

The shoe care apparatus may further configured to include a current sensor configured to measure a compressor current, and the controller is further configured to control the operation frequency of the compressor so that the compressor current is equal to or less than a preset limit current.

The controller may further configured to determine a current difference between the limit current and the compressor current at preset intervals and determine a control value of the operation frequency corresponding to the current difference by referring to a current control table stored in advance.

The shoe care apparatus may further include a second temperature sensor configured to measure a second temperature of air having passed the air outlet, and the controller is further configured to determine the outside air temperature based on the second temperature measured at a beginning of operation of the shoe care apparatus.

The shoe care apparatus may further include a sterilizer arranged in the chamber or the first duct and configured to sterilize air, and the controller is further configured to operate the fan and the sterilizer for a preset stabilization time before operation of the compressor, and operate the fan and the sterilizer for a preset cooling time after completion of a dry course in which the compressor is operated.

The controller may further configured to determine the target temperature based on a selection of a shoe type and a caring course input through the control panel.

According to an embodiment, a method of controlling a shoe care apparatus including a first duct connected to an air outlet of a chamber, a second duct connected to an air inlet of the chamber, and a holder arranged in the chamber includes determining a target temperature of air to be introduced into the chamber based on a user input obtained through a control panel; determining an operation frequency of a compressor based on the target temperature and an outside air temperature; operating the compressor at the operation frequency; measuring a first temperature of air heated by a condenser arranged in the first duct; and controlling the operation frequency of the compressor based on the target temperature and the first temperature.

The controlling of the operation frequency may include increasing or decreasing the operation frequency of the compressor for the first temperature to reach the target temperature in response to the first temperature reaching a preset first threshold temperature.

The controlling of the operation frequency may include determining a temperature difference between the target temperature and the first temperature, determining a value of variation in the temperature difference at preset intervals; and determining a control value of the operation frequency corresponding to the temperature difference and the value of variation in the temperature difference by referring to a fuzzy table.

The method of controlling the shoe care apparatus may further include stopping an operation of the compressor in response to the first temperature reaching a preset second threshold temperature and the operation frequency reaching a preset minimum frequency; and operating the compressor which has been stopped in response to the first temperature reduced below a preset third threshold temperature.

The method of controlling the shoe care apparatus may further include stopping an operation of the compressor in response to a temperature of a refrigerant measured at an inlet of an evaporator equal to or greater than a preset protection temperature; and operating the compressor which has been stopped in response to the first temperature reduced below a preset third threshold temperature.

The method of controlling the shoe care apparatus may further include measuring a compressor current by a current sensor, wherein the controlling of the operation frequency may further include controlling the operation frequency of the compressor for the compressor current to be equal to or less than a preset limit current.

The controlling of the operation frequency may include determining a current difference between the limit current and the compressor current at preset intervals; and determining a control value of the operation frequency corresponding to the current difference by referring to a current control table.

The method of controlling the shoe care apparatus may further include measuring a second temperature of air having passed the air outlet; and determining the outside air temperature based on the second temperature measured at a beginning of operation of the shoe care apparatus.

The method of controlling the shoe care apparatus may further include operating a fan and a sterilizer for a preset stabilization time before an operation of the compressor; and operating the fan and the sterilizer for a preset cooling time after a completion of a dry course in which the compressor is operated.

The determining of the target temperature of the air may be based on selecting of a shoe type and a caring course input through the control panel

### BRIEF DESCRIPTION OF THE DRAWINGS

FIG. 1 illustrates a shoe care apparatus, according to an embodiment.

FIG. 2 is a perspective view of a shoe care apparatus with a door opened, according to an embodiment.

FIG. 3 is a cross-sectional view of a shoe care apparatus viewed from the front of the shoe care apparatus, according to an embodiment.

FIGS. 4 and 5 are perspective views of a holder installed in a chamber.

FIG. 6 illustrates an installation rail to be installed in a chamber.

FIG. 7 schematically illustrates a flow of air and a flow of refrigerant in a shoe care apparatus, according to an embodiment.

FIG. 8 is a control block diagram of a shoe care apparatus, according to an embodiment.

FIG. 9 is a flowchart describing an overall operation procedure of a shoe care apparatus, according to an embodiment.

FIG. 10 is a flowchart briefly describing an operation of a compressor in a dry course.

FIG. 11 is a flowchart describing fuzzy control in a method of controlling an operation frequency of a compressor.

FIG. 12 illustrates a fuzzy table.

FIG. 13 is a graph illustrating results of fuzzy control.

FIG. 14 is a graph illustrating limits of fuzzy control.

FIG. 15 is a flowchart describing compressor switching control in a method of controlling a shoe care apparatus.

FIG. 16 is a flowchart describing compressor switching control in another embodiment.

FIG. 17 is a graph illustrating results of compressor switching control.

FIG. 18 is a flowchart describing compressor current control in a method of controlling a shoe care apparatus.

FIGS. 19 and 20 are graphs illustrating an example in which compressor current control is not applied in a heatup process.

FIG. 21 illustrates a current control table.

FIG. 22 is a table illustrating embodiments in which the operation frequency of a compressor is controlled based on an outside air temperature and a target temperature.

FIGS. 23, 24 and 25 are graphs illustrating control results of a shoe care apparatus according to a target temperature in an environment where outside air temperature is low.

FIGS. 26 and 27 are graphs illustrating control results of a shoe care apparatus according to settings of high target temperature.

FIG. 28 is a graph illustrating an example in which an operation frequency of a compressor is set to be low.

### DETAILED DESCRIPTION

Like numerals refer to like elements throughout the specification. Not all elements of embodiments of the disclosure will be described, and description of what are commonly known in the art or what overlap each other in the embodiments will be omitted. The terms as used throughout the specification, such as “˜part”, “˜module”, “˜member”, “˜block”, etc., may be implemented in software and/or hardware, and a plurality of “˜parts”, “˜modules”, “˜members”, or “˜blocks” may be implemented in a single element, or a single “˜part”, “˜module”, “˜member”, or “˜block” may include a plurality of elements.

It will be further understood that the term “connect” or its derivatives refer both to direct and indirect connection, and the indirect connection includes a connection over a wireless communication network or an electric connection through electric wires.

The terminology used herein is for the purpose of describing embodiments and does not limit the disclosure. It is to be understood that the singular forms “a,” “‘an,” and “the” include plural references unless the context clearly dictates otherwise. In the specification, the term “include”, “comprise”, “have”, etc., is used to describe existence of a feature, a number, a step, an operation, a component, a part, or any combination thereof as disclosed herein, but does not rule out including other feature(s) or component(s).

Furthermore, throughout the specification, ordinal numbers used before components are used to distinguish the components from one another, and do not imply order of arrangement, manufacturing, or importance. Descriptions shall be understood as to include any and all combinations of one or more of the associated listed items when the items are described by using the conjunctive term “˜ and/or ˜,” or the like.

The terms “forward (or front)”, “rearward (or behind)”, “left”, and “right” as herein used are defined with respect to the drawings, but the terms may not restrict the shape and position of the respective components.

The disclosure provides a shoe care apparatus and method for controlling the same, capable of keeping the air supplied into a chamber at a constant temperature by circulating the air in the closed apparatus using a heat pump cycle.

The disclosure also provides a shoe care apparatus and method for controlling the same, capable of reducing a heatup time of air supplied into a chamber by efficiently using a maximum operating frequency of a compressor without damaging a control circuit due to excessive current.

According to the disclosure, the shoe care apparatus and method for controlling the same may keep the air in a chamber at a constant temperature by circulating the air in the closed apparatus using a heat pump cycle. Accordingly, thermal damage to an object to be dried due to heat buildup in the chamber may be prevented.

According to the disclosure, the shoe care apparatus and method for controlling the same may also reduce a heatup time of air in the chamber by efficiently using a maximum frequency of a compressor without damaging a control circuit due to an excessive current.

According to the disclosure, the shoe care apparatus and method for controlling the same may also keep the environment around the apparatus pleasant because contaminated air produced in the process of drying and deodorizing an abject to be dried is not discharged to the outside.

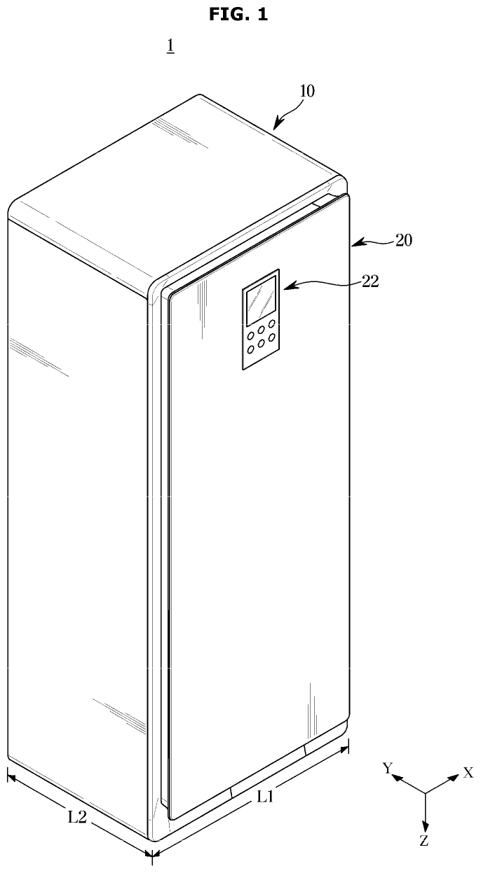

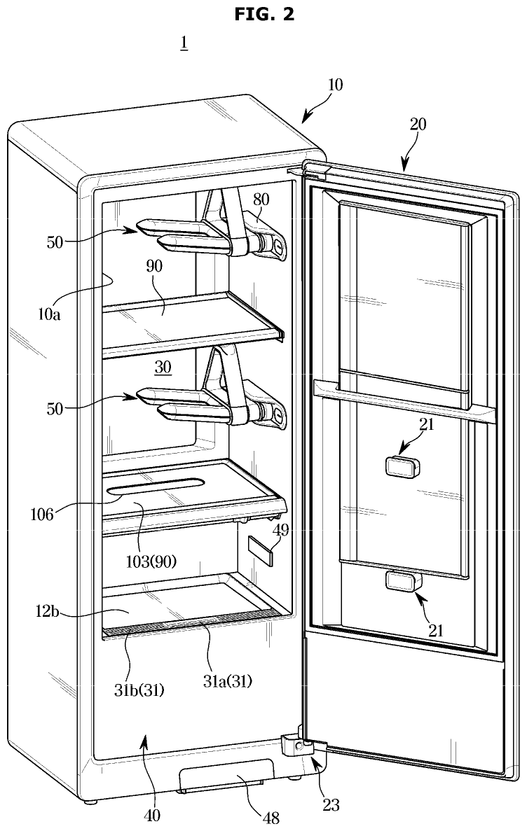

As shown in FIGS. 1 and 2, it is defined that a shoe care apparatus 1 is installed with a door 20 facing forward, and based on which, rearward, left, right, upward and downward may be defined.

Embodiments of the disclosure will now be described in detail.

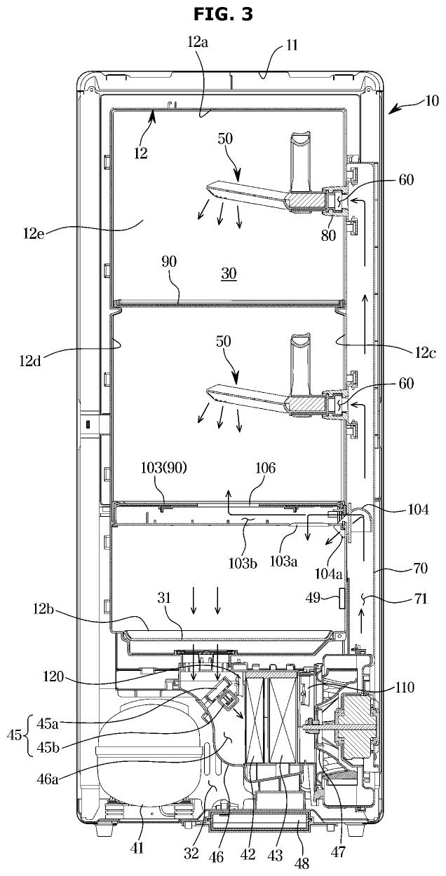

FIG. 1 illustrates a shoe care apparatus, according to an embodiment. FIG. 2 is a perspective view of a shoe care apparatus with a door opened, according to an embodiment. FIG. 3 is a cross-sectional view of a shoe care apparatus viewed from the front of the shoe care apparatus, according to an embodiment.

Referring to FIG. 1, the shoe care apparatus 1 may include a main body 10 defining the exterior and the door 20 rotationally coupled to the main body 10.

The main body 10 may be shaped like a rectangular parallelepiped with the front side open. An opening 10a may be formed on the open front of the main body 10. The door 20 is rotationally coupled with the main body 10 to open or close the open front of the main body 10. The door 20 may be coupled with the main body 10 by a hinge 23.

The main body 10 may be formed such that a front length extending in a first direction X is different from a side length extending in a second direction Y. Specifically, the front of the main body 10 may be formed in length L1 longer than length L2 of the side of the main body 10. This may make it easy to install the shoe care apparatus 1 even in a narrow entrance of the house. The front length of the main body 10 and the side length of the main body 10 may be defined as the first length L1 and the second length L2, respectively.

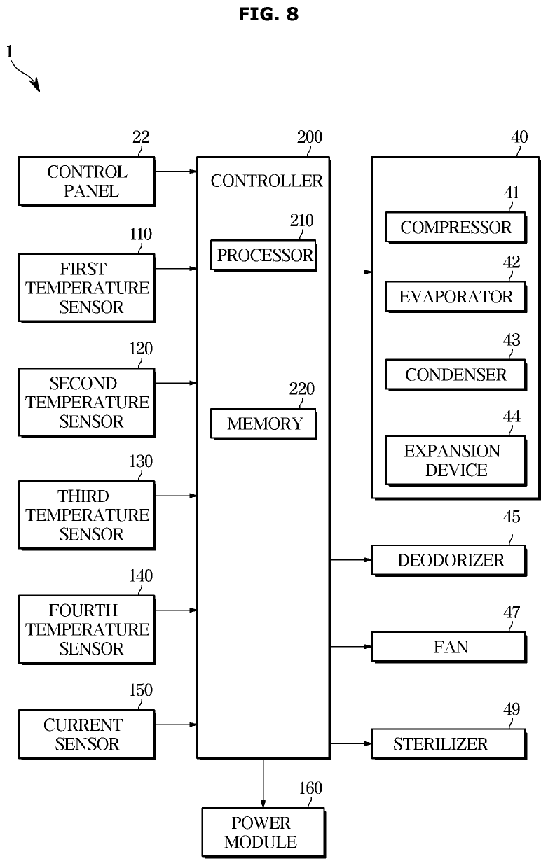

The door 20 may include a control panel 22 arranged on the front or top surface. The control panel 22 may receive various commands from the user. The control panel 22 may also display various information about operations of the shoe care apparatus 1. For example, the user may use the control panel 22 to select a type of shoes to be cared and set a suitable caring course for the shoes. Shoe types and caring courses will be described in detail in connection with FIG. 8.

The control panel 22 may include a display for displaying information about operation of the shoe care apparatus 1. The control panel 22 may also include at least one of a button or a touch screen.

Referring to FIG. 2, the door 20 may include a hanging member 21. The hanging member 21 may be arranged on one surface of the door 20 facing the interior of a chamber 30 and there may be at least one hanging member 21. The hanging member 21 may be used for the purpose of hanging a handle 55 of a holder 50. The hanging member 21 may make it easy to store the holder 50. The hanging member 21 may also be used for other purposes.

Referring to FIG. 3, the main body 10 may include an outer case 11 and an inner case 12 arranged within the outer case 11. The inner case 12 may form the chamber 30. The holder 50 that may hold shoes may be provided in the chamber 30. The inner case 12 may be referred to as a case.

The chamber 30 may form a space for receiving shoes. The chamber 30 may include a top surface 12a, a bottom surface 12b, a left surface 12c, a right surface 12d, and a rear surface 12e of the inner case 12.

The holder 50 and an installation rail 80 may be arranged in the chamber 30. The holder 50 and the installation rail 80 may be installed on the left surface 12c or the right surface 12d. Specifically, the holder 50 may be installed to show a side of the shoes when viewed from the front of the shoe care apparatus 1. For this, the side of the main body 10 may be formed in a length shorter than the length of the front of the main body 10. Positions of the holder 50 and the installation rail 80 are not limited thereto.

There may be at least one holders 50 arranged. The holder 50 may be provided in the form that may be inserted into the shoes. Furthermore, the holder 50 may be detachable from the chamber 30. Specifically, the holder 50 may be coupled to the installation rail 80 arranged on a side surface of the chamber 30, and may be detachable from the installation rail 80. For example, the holder 50 may be inserted to the installation rail 80 along the second direction Y. As the holder 50 is detachable, the space in the chamber 30 may be efficiently used depending on the size of the shoes.

The chamber 30 may include an air inlet 60 and an air outlet 31. The air inlet 60 may be formed on a side wall of the inner case 12. For example, the air inlet 60 may be formed on the left surface 12c of the chamber 30. The air inlet 60 may be provided in the plural. Air heated by a condenser 43 may be supplied into the chamber 30 through the air inlet 60. The air inlet 60 may be formed in various shapes. For example, the air inlet 60 may have a circular, rectangular, or polygonal shape.

The air outlet 31 may be arranged on the bottom surface 12b of the chamber 30. For example, the air outlet 31 may be arranged on a front side of the bottom surface 12b of the chamber. The air in the chamber 30 may flow to a first duct 46 through the air outlet 31. The air outlet 31 may be comprised of a center hole 31a and a grill 31b having a plurality of side holes.

A machine room 32 may be arranged under the chamber 30. In the machine room 32, a compressor 41, an evaporator 42, the condenser 43, an expansion device 44, a deodorizer 45, the first duct 46, a fan 47, a first temperature sensor 110 and a second temperature sensor 120 may be arranged. Furthermore, there may be a sterilizer 49 arranged in the chamber 30 or the machine room 32. In FIGS. 2 and 3, the sterilizer 49 is shown as being arranged in the chamber 30.

The compressor 41, the evaporator 42, the condenser 43 and the expansion device 44 may be defined as a heat pump device 40. The heat pump device 40 may dehumidify and heat air circulating in the chamber 30. The heat pump device 40 may supply the heated air into the chamber 30.

Furthermore, the machine room 32 may be further equipped with a third temperature sensor 130 arranged on an inlet side of the evaporator 42, a fourth temperature sensor 140 arranged on an outlet side of the compressor 41, and a current sensor 150 for measuring a compressor current applied to the compressor 41.

The first duct 46 is a duct located under the chamber 30, which may also be referred to as a lower duct. The first duct 46 may be connected to the air outlet 31 of the chamber 30 to form a first flow path 46a that guides the air having passed the air outlet 31 to the fan 47. The first duct 46 may also be connected to a second duct 70 arranged within a side of the main body 10. The second duct 70 may be referred to as an upper duct.

The second duct 70 may be provided outside of a side wall of the inner case 12 in the second direction Y of the shoe care apparatus 1. The second duct 70 may have one end connected to at least one supply port 60 and the other end connected to the first duct 46. The second duct 70 may form a second flow path 71 for guiding air to the supply port 60.

The evaporator 42 and the condenser 43 may be arranged in the first duct 46. The evaporator 42, the condenser 43 and the fan 47 may be arranged in the first direction X. The evaporator 42 may be located farther upstream of the air flow than the condenser 43.

The fan 47 may be arranged between the heat pump device 40 and the chamber 30 to circulate air. The fan 47 may be rotated based on preset rotations per minute (rpm). Specifically, the fan 47 may suck in air brought into the first duct 46 and discharge the air to the second duct 70. The air brought into the first duct 46 through the air outlet 31 may be dried while passing the evaporator 42 of the heat pump device 40, heated while passing the condenser 43, and discharged back to the chamber 30 through the second duct 70 and the supply port 60.

The fan 47 may include a motor (not shown) and a blade (not shown). The blade may be rotated by operation of the motor, and accordingly, air is moved. The fan 47 may have various types. For example, the fan 47 may be provided as a centrifugal fan.

Furthermore, the deodorizer 45 may be arranged in the first duct 46. The deodorizer 45 may include a deodorizing filter 45a and an ultraviolet (UV) light emitting diode (LED) 45b. The deodorizing filter 45a and the UV LED 45b may be arranged to be near the air outlet 31 of the chamber 30. The UV LED 45b may irradiate light to the deodorizing filter 45a to remove odor of the air. For example, the deodorizing filter 45a may include at least one of a ceramic filter, a photocatalyst filter or an activated carbon filter.

The sterilizer 49 may be further arranged in the chamber 30 or the first duct 46. The sterilizer 49 may remove germs contained in the air. The sterilizer 49 may include at least one of an ultraviolet lamp, an ultraviolet LED, a Zenon lamp, an ozone generator or a disinfectant spray.

A drain tub 48 may be arranged in a lower portion of the main body 10, i.e., underneath the machine room 32. The drain tub 48 may store condensate water produced by the evaporator 42. The drain tub 48 is detachable from the main body 10.

At least one shelf 90 may be arranged in the chamber 30. Shoes may be placed on the shelf 90. The shelf 90 may further include a duct shelf 103. The duct shelf 103 may form a flow path 103b inside and include a hole 103a on the bottom surface. The air rising from the fan 47 through the second duct 70 may be discharged into the chamber 30 through the hole 103a of the duct shelf 103. Furthermore, there may be a hole 106 formed even on the top surface of the duct shelf 103.

A side surface of the duct shelf 103 may be connected to a circular duct 104 arranged in the second duct 70. Air may be discharged into the chamber 30 through a nozzle 104a of the circular duct 104. The air may pass the circular duct 104 and then may be supplied to the duct shelf 103. The circular duct 104 may have various shapes. For example, the circular duct 104 may have a fan shape.

The first temperature sensor 110 may measure first temperature of air heated by the condenser 43. Hereinafter, the temperature of air measured by the first temperature sensor 110 is defined as the first temperature. The first temperature sensor 110 may be arranged in a flow path between the condenser 43 and the fan 47. A controller 200 of the shoe care apparatus 1 may control an operation frequency of the compressor 41 based on the first temperature measured by the first temperature sensor 110.

The second temperature sensor 120 may measure the temperature of air at the air outlet 31 of the chamber 30. The second temperature sensor 120 may be arranged in a flow path between the air outlet 31 and the deodorizing filter 45a or between the deodorizing filter 45a and the evaporator 42. Hereinafter, the temperature of the air measured by the second temperature sensor 120 is defined as the second temperature. The controller 200 of the shoe care apparatus 1 may determine an outside air temperature based on the second temperature measured by the second temperature sensor 120 at the start of operation of the shoe care apparatus 1.

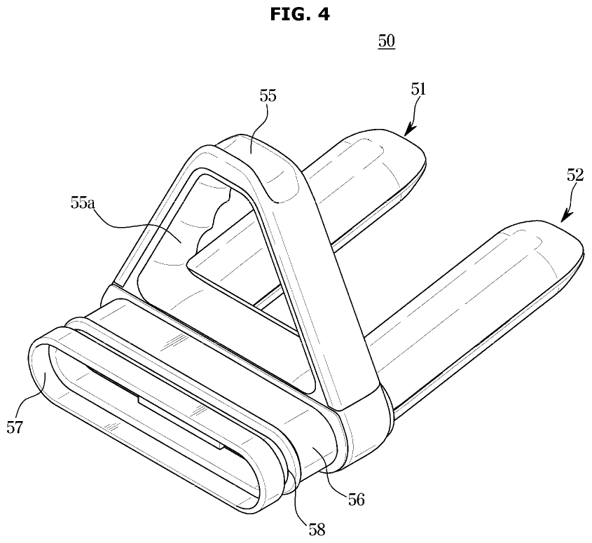

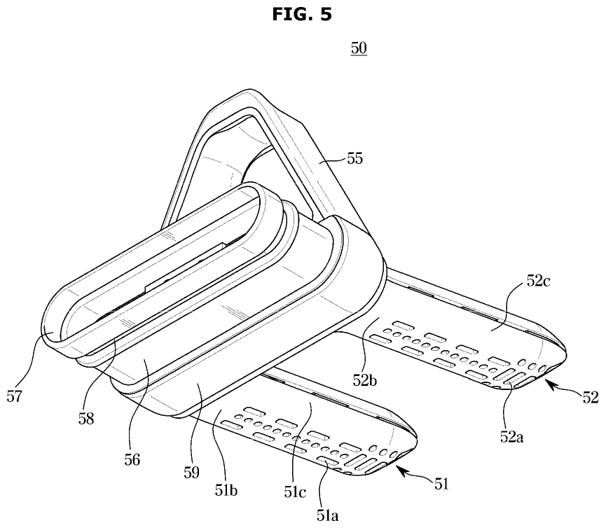

FIGS. 4 and 5 are perspective views of a holder installed in a chamber.

Referring to FIGS. 4 and 5, the holder 50 may include support frames 51 and 52, the holder 55, a support body 56 and a coupler 57. The support body 56 may connect the handle 55, the coupler 57 and the support frames 51 and 52.

The support frames 51 and 52 may include the first support frame 51 and the second support frame 52. The first support frame 51 and the second support frame 52 may protrude from a side surface of the chamber 30 in the first direction X and may be spaced apart from each other in the second direction Y. Although two support frames 51 and 52 are shown, there may be one or more than two support frames. The first support frame 51 and the second support frame 52 may be spaced apart from each other along the second direction Y to hold a plurality of shoes.

In the meantime, the support frames 51 and 52 may be sloped at a certain angle to prevent the shoes caught from falling out. In other words, the support frames 51 and 52 may be formed to incline upward with respect to the bottom surface 12b of the chamber 30. Accordingly, the shoes caught by the holder 50 may not fall out.

The handle 55 may make the holder 50 easily moved or detached. The user may grip the handle 55 to move the holder 50. Furthermore, the user may use the handle 55 to easily install the holder 50 on the installation rail 80. The handle 55 may be formed in various shapes. For example, the handle 55 be formed in a triangular shape. Furthermore, a grip member 55a may be formed on the handle 55. The user may use the grip member 55a to easily grip the handle 55.

The coupler 57 may be connected to the air inlet 60 to guide the air supplied through the second duct 70 to the support frames 51 and 52. Although the coupler 57 is shown as having the form of an oval hollow, it is not limited thereto and may have other various shapes.

Referring to FIG. 5, the support frames 51 and 52 of the holder 50 may include nozzles 51a and 52a, respectively. The first support frame 51 may include the first nozzle 51a, and the second support frame 52 may include the second nozzle 52a. The nozzle 51a and 52a may be formed on at least one of the bottom surface 51b and 52b or a side surface 51c and 52c. The nozzle 51a and 52a may be provided in various shapes. For example, the nozzle 51a and 52a may be shaped like a circle, an oval, or a rectangle. The heated air may be supplied into the chamber 30 through the nozzles 51a and 52a.

The holder 50 may further include a fastening groove 58. A fixing projection 84 of the installation rail 80 may be inserted to the fastening groove 58 to fix the holder 50. The holder 50 may further include a reinforcing member 59. The reinforcing member 59 may be connected to the handle 55 to reinforce the support body 56.

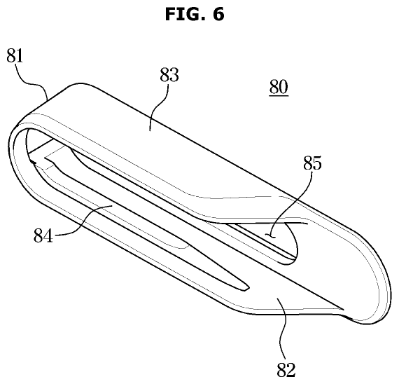

FIG. 6 illustrates an installation rail to be installed in a chamber.

Referring to FIG. 6, one end 81 of the installation rail 80 is closed to prevent the holder 50 from falling out, and the other end 82 of the installation rail 80 has an open form to receive the holder 50. The installation rail 80 may include a fixing frame 83 and the fixing projection 84.

The fixing frame 83 extends from the one end 81 to the other end 82 of the installation rail 80 and may receive the coupler 57 of the holder 50. The fixing projection 84 may be inserted to the fastening groove 58 of the holder 50. Accordingly, the holder 50 may be fixed to the installation rail 80. The holder 50 is detachable from the installation rail 80.

Furthermore, the installation rail 80 may include an air hole 85. The air brought in through the second duct 70 and the air inlet 60 of the chamber 30 may be supplied to the holder 50 through the air hole 85 of the installation rail 80. Specifically, the air brought in from the air inlet 60 may be supplied to the support frames 51 and 52 of the holder 50 through the air hole 85 and sprayed into the chamber 30 through the nozzles 51a and 52a.

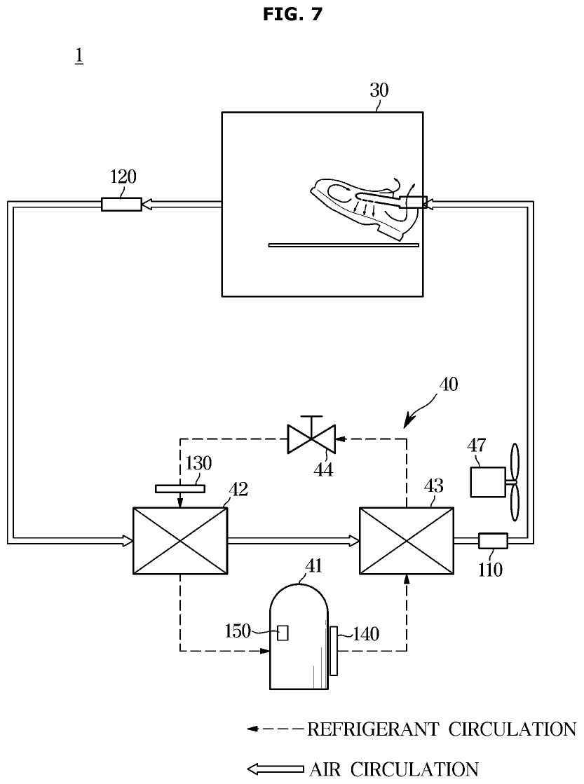

FIG. 7 schematically illustrates a flow of air and a flow of refrigerant in a shoe care apparatus, according to an embodiment.

Referring to FIG. 7, the shoe care apparatus 1 in the embodiment includes the chamber 30 for receiving an object to be dried S, the heat pump device 40 for dehumidifying and heating the air in the chamber 30 to dry the object S, the first temperature sensor 110 for measuring the first temperature of the air heated by the condenser 43, the second temperature sensor 120 for measuring the second temperature of the air having passed through the air outlet 31 of the chamber 30, and the fan 47 arranged between the heat pump device 40 and the chamber 30 for circulating the air.

The heat pump device 40 includes the compressor 41, the condenser 43, the expansion device 44 and the evaporator 42. The compressor 41, the condenser 43, the expansion device 44 and the evaporator 42 may be connected by refrigerant pipes to each other to form a heat pump cycle, and the refrigerant may circulate in the heat pump cycle while flowing in the refrigerant pipes.

The compressor 41 compresses low-temperature and low-pressure vapor-phase refrigerant and discharges high-temperature and high-pressure vapor-phase refrigerant. The discharged vapor-phase refrigerant may flow into the condenser 43, and the condenser 43 may condense the high-temperature and high-pressure vapor-phase refrigerant to high-pressure liquid-state or approximately liquid-state refrigerant equal to or lower than a condensation temperature. The high-pressure liquid-phase or approximately liquid-state refrigerant having passed the condenser 43 is expanded and decompressed by an expansion device 44, and the low-temperature and low-pressure two-phase refrigerant having passed the expansion device 44 flows into the evaporator 42. The two-phase refrigerant may be evaporated to the vapor-state refrigerant in the evaporator 42.

The chamber 30 and the heat pump device 40 may be connected by the first duct 46 and the second duct 70, and the air in the chamber 30 may be moved through the ducts and may circulate through the pump device 40 and the chamber 30.

The hot and humid air in the chamber 30 may exchange heat with the refrigerant while passing the evaporator 42. Specifically, the low-temperature and low-pressure two-phase refrigerant brought into the evaporator 42 may be evaporated into the vapor-phase refrigerant by absorbing heat from the hot and humid air passing the evaporator 42, and the hot and humid air passing the evaporator 42 is cooled and dehumidified at the same time into cool and dry air.

The cool and dry air having passed the evaporator 42 flows into the condenser 43, and heat may be exchanged between the high-temperature and high-pressure vapor-phase refrigerant and the cool and dry air in the condenser 43. The high-temperature and high-pressure vapor-phase refrigerant may release heat while being condensed into the liquid-phase or approximately liquid-phase refrigerant, and the cool and dry air may be heated by absorbing the heat released in the condensation process of the refrigerant.

The hot and dry air having passed the condenser 43 may flow back into the chamber 30. With this air circulation cycle, the shoes S received in the chamber 30 may be dried.

The expansion device 44 may be implemented with at least one of a capillary tube or an electric expansion valve that may control opening degrees based on an electric signal, and the compressor 41 may be implemented as a frequency changeable inverter compressor. The frequency of the compressor 41 refers to revolutions per second of a motor connected to a compression room of the compressor 41. The compressor 41 may operate at a preset starting frequency at the start of a dry course, and afterward, to increase the temperature, the compressor 41 may operate at an operating frequency. In the meantime, the compressor 41 may operate within a range from a minimum frequency and a maximum frequency. The minimum operation frequency and the maximum operation frequency may be set in advance depending on the design.

The shoe care apparatus 1 may further include the third temperature sensor 130 arranged on the inlet side of the evaporator 42 and the fourth temperature sensor 140 arranged on the outlet side of the compressor 41. The third temperature sensor 130 and the fourth temperature sensor 140 may be installed outside or inside the refrigerant pipe to measure temperature of the refrigerant circulating in the heat pump cycle. Specifically, the third temperature sensor 130 may measure temperature of the refrigerant flowing into the evaporator 42 and the fourth temperature sensor 140 may measure temperature of the refrigerant discharged from the compressor 41.

The shoe care apparatus 1 may further include the current sensor 150 for measuring a compressor current applied to the compressor 41. The current sensor 150 may measure power consumed by the compressor 41.

FIG. 8 is a control block diagram of a shoe care apparatus, according to an embodiment.

Referring to FIG. 8, the shore care apparatus 1 may include the control panel 22, the heat pump device 40, the deodorizer 45, the fan 47, the sterilizer 49, the first temperature sensor 110, the second temperature sensor 120, the third temperature sensor 130, the fourth temperature sensor 140, the current sensor 150, a power module 160, and the controller 200. Although not shown, the shoe care apparatus 1 may further include a communication device (not shown) for transmitting or receiving data to or from an external device. The controller 200 may be electrically connected to the components of the shoe care apparatus 1 to control operations of the respective components.

The power module 160 may supply power to the components of the shoe care apparatus 1. The power module 160 may be implemented with a printed circuit board and a power circuit mounted on the printed circuit board. For example, the power module 160 may include a power circuit board, and a capacitor, a coil, a resistor, a processor, etc., which are mounted on the power circuit board.

The controller 200 may include a memory 220 for storing a program, instructions and data for controlling operations of the shoe care apparatus 1, and a processor 210 for generating control signals to control the operations of the shoe care apparatus 1 based on the program, instructions and data stored in the memory 220. The controller 200 may be implemented with a control circuit having the processor 210 and the memory 220 mounted thereon. Furthermore, the controller 200 may include a plurality of processors and a plurality of memories.

The processor 210 may include logic circuits and operation circuits in hardware. The processor 210 may process the data according to the program and/or instructions provided from the memory 220 and generate a control signal based on the processing result. For example, when the user inputs a command to select a caring course by manipulating the control panel 22, the shoe care apparatus 1 may perform shoe care corresponding to the selected caring course.

The memory 220 may include a volatile memory such as a static random access memory (S-RAM), dynamic RAM (D-RAM), etc., for temporarily storing data, and a non-volatile memory such as a read only memory (ROM), an erasable programmable ROM (EPROM), an electrically erasable programmable (ROM) (EEPROM), etc., for storing data for a long time.

The control panel 22 may be arranged on the door 20 as described above in connection with FIG. 1. Although the control panel 22 is shown as being arranged on the front surface of the door 20, it is not limited thereto but may be arranged in various positions. The controller 200 may determine a target temperature based on selecting of a shoe type and a caring course input through the control panel. Furthermore, the controller 200 may determine operation time based on the selecting of a shoe type and a caring course.

The user may use the control panel 22 to select a type of shoes to be cared for. For example, the control panel 22 may provide at least one of a shoe type menu to allow a shoe type to be selected or a caring course menu to allow a caring course to be selected. Shoe types may include types according to the purpose or the shape such as dress shoes, running shoes, hiking boots, sandals, rain boots, etc. The shoe types may also include types according to the material such as leather, cotton, nylon, a mixing material, enamel, suede, neoprene, etc.

The controller 200 may determine the target temperature of the air to be supplied into the chamber 30 based on the shoe type. As different types of shoes have different characteristics, the target temperature for caring the shoes may be set differently according to the characteristics of the shoes. For example, for shoes of mixing materials, a low target temperature of 300 or higher and less than 380 may be set. For shoes of a leather material, a medium target temperature of 380 or higher and less than 430 may be set. For shoes of a cotton material, a high target temperature of 430 or higher and less than 600 may be set. In another example, when two or more types of shoes are to be cared for or the shoes contains moisture, a target temperature for dehumidification may be set to less than 400, and a target temperature for deodorization may be set to 400 or higher and less than 600. With this, damage to the shoes may be prevented. Furthermore, when both dehumidification and deodorization are required, deodorization may be performed after dehumidification. Specifically, the moisture contained in the shoes is removed at a low temperature and deodorization is then performed at a high temperature, thereby minimizing the damage to the shoes.

Furthermore, the user may use the control panel 22 to set a suitable caring course for the shoes. The controller 200 may determine an operation time of the shoe care apparatus 1 based on the caring course. For example, the caring course may include at least one of a standard course, a quick course, an intense course or a clean storage course. The standard course is a default caring course, and may be defined as a caring course in which the shoe care apparatus 1 is operated for a standard time (e.g., 30 minutes) for which dehumidification and deodorization effects are normally exerted. The quick course may be defined as a caring course that may exert minimum dehumidification and deodorization effects within a shorter time than in the standard course. The intense course may be defined as a caring course that may exert maximum dehumidification and deodorization effects by being operated for a longer time than in the standard course. Furthermore, the clean storage course may be defined as a caring course for keeping the shoes for a long time. As such, various caring courses may be suitably applied to various shoes, thereby increasing convenience of shoe caring and user satisfaction.

The controller 200 may determine outside air temperature based on the second temperature measured by the second temperature sensor 120 at the start of operation of the shoe care apparatus 1. Before the start of operation of the shoe care apparatus 1, the door 20 may be opened to put shoes into the chamber 30. In this case, the temperature of the air in the chamber 30 is equivalent to the outside air temperature. Furthermore, at the start of operation of the shoe care apparatus 1, the air in the chamber 30 is in a non-heated state. Hence, at the start of operation of the shoe care apparatus 1, the outside air temperature may be determined by using the second temperature sensor 120 located at the air outlet 31 of the chamber 30.

The controller 200 may determine an operation frequency of the compressor 41 based on the target temperature and the outside air temperature, and operate the compressor 41 at the determined operation frequency. The target temperature and the outside air temperature are factors that have big influences on determination of an operation frequency of the compressor 41. For example, when the outside air temperature is low, the operation frequency of the compressor 41 may be set to a high value to force the temperature of the air supplied into the chamber 30 to quickly reach the target temperature. The larger the difference between the outside air temperature and the target temperature, the higher value the operation frequency of the compressor 41 may be set to. On the other hand, when the difference between the target temperature and the outside air temperature is small (e.g., when the difference between the target temperature and the outside air temperature is 10□ or less), the operation frequency of the compressor 41 may be set to a low value. It is because when the temperature in the chamber 30 rises suddenly, it may exceed the target temperature.

Furthermore, the controller 200 may control the operation frequency of the compressor 41 based on the temperature of the air heated by the condenser 43 and the target temperature. Specifically, the controller 200 may control the operation frequency of the compressor 41 based on the first temperature measured by the first temperature sensor 110. When the compressor 1 is operated at a fixed operation frequency for the whole operation time, the temperature of the air circulating in the closed shoe care apparatus 1 constantly rises, failing to maintain the constant target temperature. To prevent this, the operation frequency of the compressor 41 needs to be controlled.

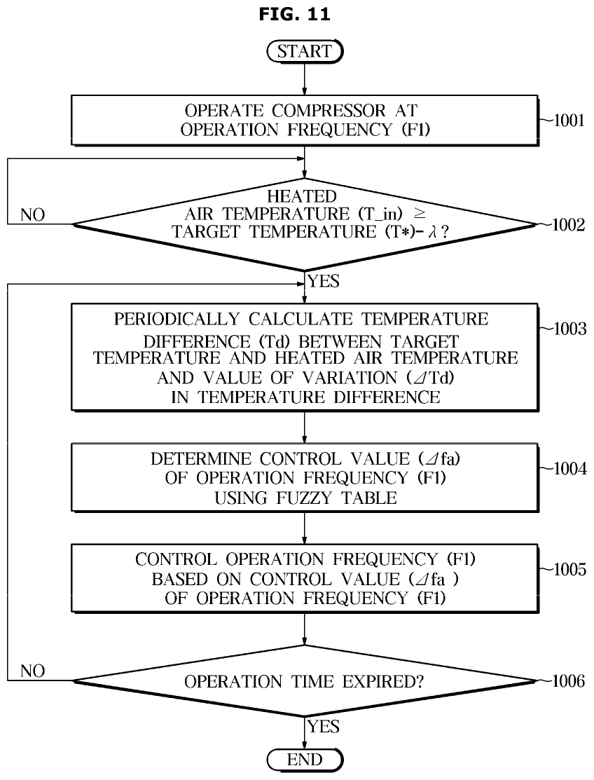

To maintain the air supplied into the chamber 30 at the target temperature, the controller 200 of the shoe care apparatus 1 may perform fuzzy control. The fuzzy control refers to a control method of periodically controlling the operation frequency of the compressor 41 for the first temperature of the air heated by the condenser 43 to follow the target temperature. The controller 200 may increase or decrease the operation frequency of the compressor 41 for the first temperature to follow the target temperature in response to the first temperature reaching a preset first threshold temperature. The controller 200 may use a fuzzy table stored in advance to determine a control value of the operation frequency. The fuzzy control will be described in detail in connection with FIG. 11.

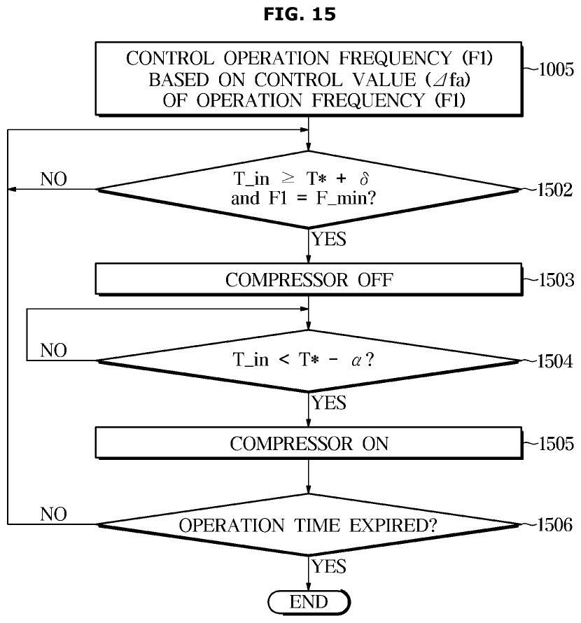

Furthermore, to complement the limit of the fuzzy control, the controller 200 may further perform compressor switching control. The compressor switching control refers to a method of controlling on or off switching of the compressor. The compressor switching control will be described in detail in connection with FIG. 15.

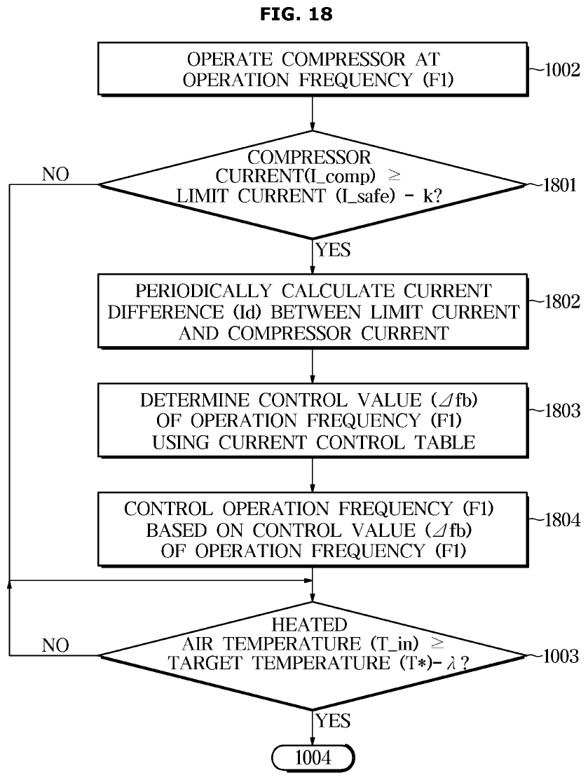

Moreover, to prevent damage to the compressor 41 due to an excessive current, the controller 200 may perform compressor current control. The compressor current control refers to a control method of controlling the current applied to the compressor 41 by periodically controlling the operation frequency of the compressor 41 based on a value of current or power applied to the compressor 41. In other words, the controller 200 may control the operation frequency of the compressor 41 for the compressor current to be equal to or less than a preset limit current. The controller 200 may use a current control table stored in advance to determine a control value of the operation frequency. The compressor current control will be described in detail in connection with FIG. 18.

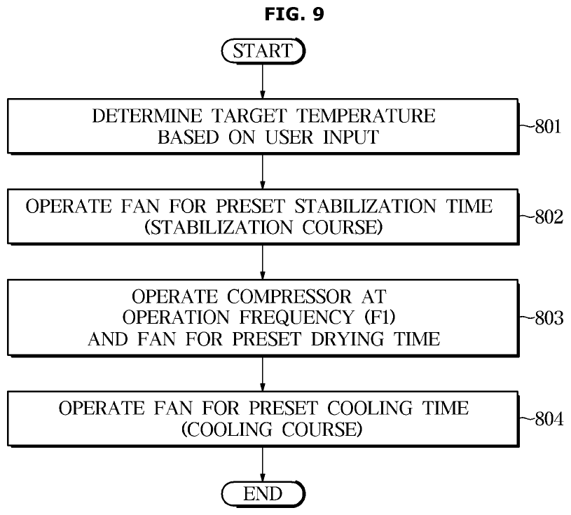

FIG. 9 is a flowchart describing an overall operation procedure of a shoe care apparatus, according to an embodiment.

Referring to FIG. 9, the overall operation procedure of the shoe care apparatus 1 may include determining a target temperature, and performing a stabilization course, a dry course and a cooling course. Through this procedure, the shoes placed in the chamber 30 may be dried and deodorized.

First, the controller 200 may determine a target temperature of air to be supplied into the chamber 30 based on a user input obtained by the control panel 22, in 801. With the determining of the target temperature, an operation time of the shoe care apparatus 1 may be determined as well. As described above, the target temperature may be determined based on selecting of a shoe type input through the control panel 22, and the operation time may be determined based on selecting of a caring course. An operation frequency of the compressor 41 may also be determined.

The controller 200 may perform the stabilization course to operate the fan 47 for a preset stabilization time, in 802. The controller 200 may operate at least one of the deodorizer 45 and the sterilizer 49 as well as the fan 47. In the stabilization course, the compressor 41 is not operated. Sudden application of a heavy load to the power module 160 and the controller 200 may be prevented through the stabilization course. Furthermore, whether the fan 47 has a failure may be detected.

Subsequently, the controller 200 may perform a dry course to operate the compressor 41 at operation frequency F1 for a preset drying time and operate the fan 47 in connection with the operation of the compressor 41, in 803. The dry course includes processes of increasing temperature of the air in the chamber 30 by operating the compressor 41 at the operation frequency F1 and maintaining the target temperature by controlling the operation frequency F1 of the compressor 41 when the temperature of the air reaches the target temperature.

The controller 200 may then perform a cooling course to operate the fan 47 for a preset cooling time, in 804. The controller 200 may operate at least one of the deodorizer 45 and the sterilizer 49 as well as the fan 47. In the cooling course, the compressor 41 is not operated. Through the cooling course, temperature in the chamber 30 may be forced to decrease and the dried shoes may be cooled. Accordingly, after completion of the operation of the shoe care apparatus 1, the user may safely take out the shoes.

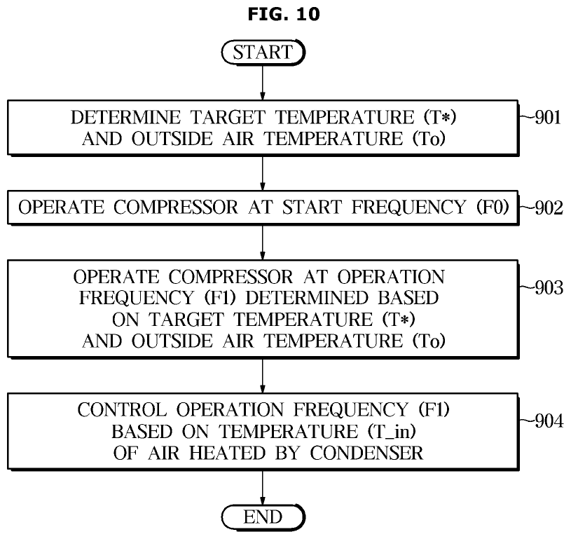

FIG. 10 is a flowchart briefly describing an operation of a compressor in a dry course.

Referring to FIG. 10, the controller 200 of the shoe care apparatus 1 may determine a target temperature T* and an outside air temperature To, in 901. The target temperature T* may be determined based on a user input, and the outside air temperature To may be determined based on the second temperature measured by the second temperature sensor 120 at the start of operation of the shoe care apparatus 1.

The controller 200 may first operate the compressor 41 for a certain time at a start frequency F0, in 902. The controller 200 may determine an operation frequency F1 of the compressor 41 based on the target temperature T* and the outside air temperature To, and operate the compressor 41 at the determined operation frequency F1, in 903.

In the meantime, the process of operating the compressor 41 at the start frequency F0 may be omitted. In other words, the compressor 41 may be operated based on the operation frequency F1 from the beginning of the dry course.

Subsequently, the controller 200 may control the operation frequency of the compressor 41 based on temperature T_in of the air heated by the condenser 43 and the target temperature T*, in 904.

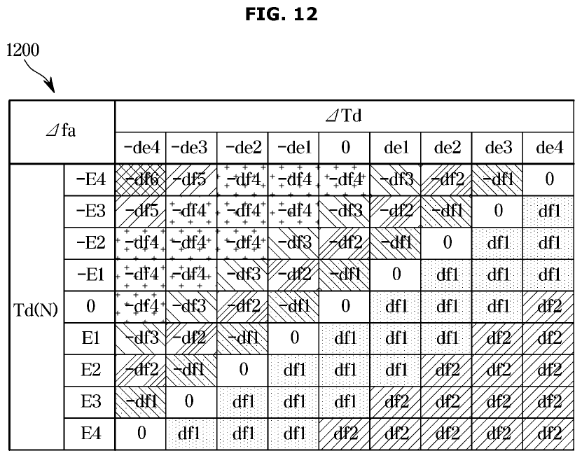

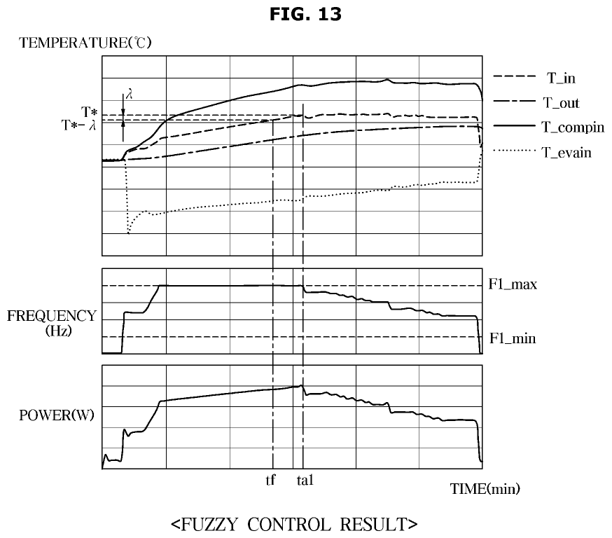

FIG. 11 is a flowchart describing fuzzy control in a method of controlling operation frequency of a compressor. FIG. 12 illustrates a fuzzy table. FIG. 13 is a graph illustrating a result of fuzzy control.

Referring to FIGS. 11 and 12, the controller 200 operates the compressor 41 at the operation frequency F1 in 1001, and checks the temperature T in of the air heated by the condenser 43 in 1002. Referring to FIG. 13, the operation frequency F1 may be set to a maximum frequency F1_max of the compressor 41. The temperature of the air heated by the condenser 43 is measured by the first temperature sensor 110 and defined as the first temperature T_in. The controller 200 checks whether the first temperature T_in reaches a preset first threshold temperature T*−λ.

When the first temperature T_in reaches the first threshold temperature T*−λ, fuzzy control is started. As shown in FIG. 13, the fuzzy control is started at time tf at which the first temperature T_in reaches the first threshold temperature T*−λ. Specifically, the controller 200 may increase or decrease the operation frequency F1 of the compressor 41 for the first temperature T_in to follow the target temperature T* when the first temperature T_in reaches the preset first threshold temperature T*−λ. The controller 200 may use a fuzzy table 1200 stored in advance to determine a control value Δfa of the operation frequency F1, and control the operation frequency F1 of the compressor 41 based on the control value Δfa.

Specifically, the controller 200 may calculate a temperature difference Td(N) between the target temperature T* and the first temperature T_in and a value of variation ΔTd in temperature difference at preset intervals, in 1003. The value of variation ΔTd in temperature difference may be calculated by subtracting a previous temperature difference Td(N-1) from the current temperature difference Td(N). The controller 200 may determine a control value Δfa corresponding to the value of variation ΔTd in temperature difference and the temperature difference Td(N) by referring to the fuzzy table 1200, in 1004. For example, in FIG. 12, when the current temperature difference Td(N) is E1 and the value of variation ΔTd in temperature difference is calculated to be −dE2, the control value Δfa of the operation frequency F1 may be determined to be −df1. The control value Δfa as mentioned in the fuzzy control may be referred to as a first control value.

The controller 200 may control the operation frequency F1 based on the control value Δfa, in 1005. Specifically, controlling of the operation frequency F1 may be performed by adding the control value Δfa to the previous operation frequency F1(N-1). For example, when the previous operation frequency F1(N-1) is 40 Hz, the operation frequency F1 may be reduced to 40 Hz-df1.

In the meantime, the controller 200 may determine an elapsed time from the start of operation of the shoe care apparatus 1 and stop operating the shoe care apparatus 1 when the operation time is expired, in 1006.

As shown in FIG. 13, it is determined that the temperature T_in of the air flowing into the chamber 30 is kept at the target temperature T* from a point of time ta1 when reaching the target temperature T*. As such, the shoe care apparatus 1 in an embodiment may maintain the constant temperature T_in of the air supplied into the chamber 30 to follow the target temperature T* by suitably controlling the frequency of the compressor 41.

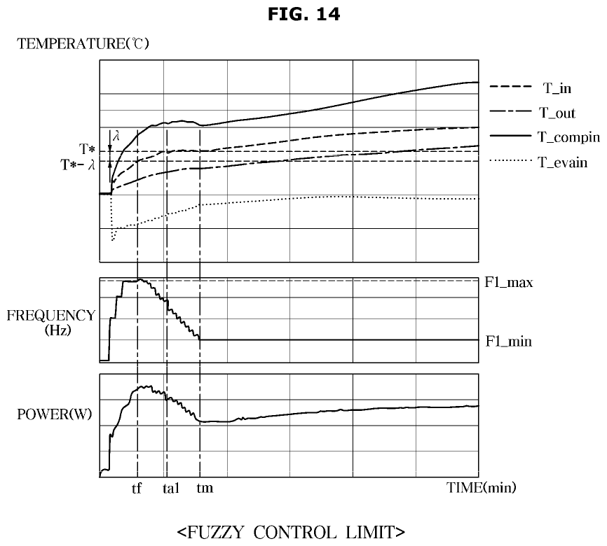

FIG. 14 is a graph illustrating limits of fuzzy control.

Referring to FIG. 14, the compressor 41 may operate within a range between a minimum frequency F1_min and a maximum frequency F1_max. However, the temperature T_in in the chamber 30 keeps rising and exceeds the target temperature T* even though the operation frequency F1 decreases after the temperature T_in in the chamber 30 reaches the target temperature T*. As shown in FIG. 14, even though the operation frequency F1 is reduced to the minimum frequency F1_min at a point of time tm, it is seen that the temperature T_in in the chamber 30 continues to rise. This problem may occur when the outside air temperature is high or when the target temperature is high. In this case, the target temperature is not maintained only by controlling the frequency of the compressor 41. Hence, to complement the limit of the fuzzy control, compressor switching control may be performed.

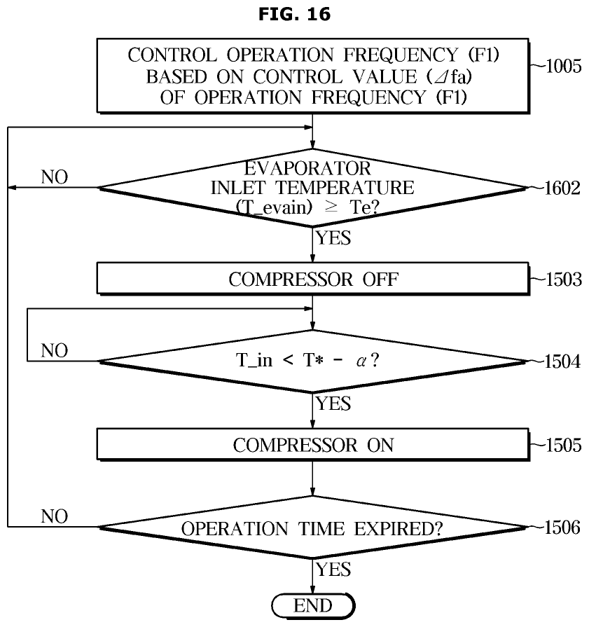

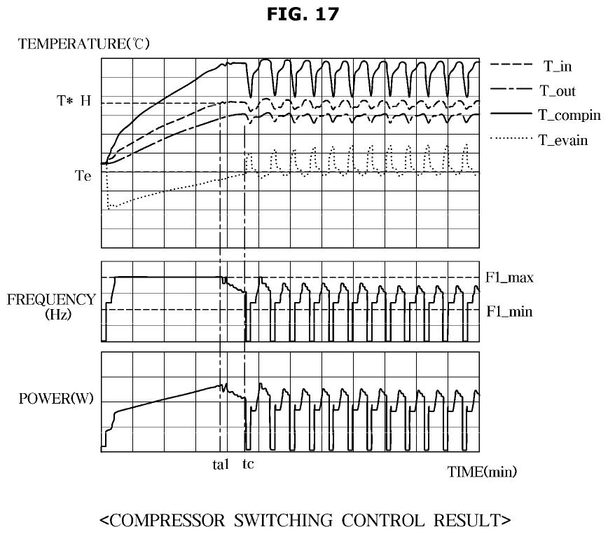

FIG. 15 is a flowchart describing compressor switching control in a method of controlling a shoe care apparatus. FIG. 16 is a flowchart describing compressor switching control in another embodiment. FIG. 17 is a graph illustrating results of compressor switching control.

In FIGS. 15 and 16, step 1005 is equivalent to what is described above. That is, the controller 200 may control the operation frequency F1 of the compressor 41 based on the control value Δfa, in 1005.

Referring to FIG. 15, the controller 200 may check whether the first temperature T_in measured by the first temperature sensor 110 reaches a preset second threshold temperature T*+δ, in 1502. The first temperature reaching the second threshold temperature includes the first temperature being equal to or higher than the second threshold temperature. The controller 200 may stop operation of the compressor 41 when the first temperature T_in measured by the first temperature sensor 110 reaches the preset second threshold temperature T*+δ and the operation frequency F1 of the compressor 41 reaches the preset minimum frequency F_min, in 1502 and 1503.

In another embodiment, referring to FIG. 13, the controller 200 may check whether temperature T_evain of the refrigerant measured by the third temperature sensor 130 arranged on the inlet side of the evaporator 42 reaches the preset protection temperature Te, in 1602. The temperature T_evain of the refrigerant reaching the protection temperature Te includes the temperature T_evain of the refrigerant being equal to or higher than the protection temperature Te. The controller 200 may stop operation of the compressor 41 when the temperature T_evain of the refrigerant measured by the third temperature sensor 130 arranged on the inlet side of the evaporator 42 is equal to or higher than the preset protection temperature Te, in 1602 and 1503.

When the temperature T_evain of the refrigerant moving to the compressor 41 from the evaporator 42 is higher than a preset maximum temperature value, the compressor 41 may be damaged. Hence, when the temperature T_evain at the inlet of the evaporator reaches the protection temperature Te, the compressor 41 is forced to be off to protect the compressor 41. The protection temperature Te may be equal to or lower than a preset maximum temperature of the refrigerant.

With the passage of certain time after the operation of the compressor 41 is stopped, the first temperature T_in may be reduced below the third threshold temperature T*−α. The certain time may be preset and defined as a compressor stabilization time (e.g., 3 minutes). When the first temperature T in is reduced below the third threshold temperature T*−α, the controller 200 of the shoe care apparatus 1 may reoperate the compressor 41, in 1504 and 1505. When the temperature of the air supplied into the chamber 30 is lower than the target temperature due to the termination of operation of the compressor 41, control to maintain the target temperature may be performed by reoperating the compressor 41.

The controller 200 may determine an elapsed time from the start of operation of the shoe care apparatus 1 and stop operating the shoe care apparatus 1 when the operation time is expired, in 1506.

Referring to FIG. 17, when a high target temperature T*_H is set, it is seen that the target temperature is maintained within a certain range according to compressor switching control. Specifically, the temperature T_in of the air flowing into the chamber 30 reaches the target temperature T*_H at a point of time ta1, and the temperature T_evain at the evaporator inlet reaches the protection temperature Te at a later point of time tc. Accordingly, on or off of the compressor 41 is switched.

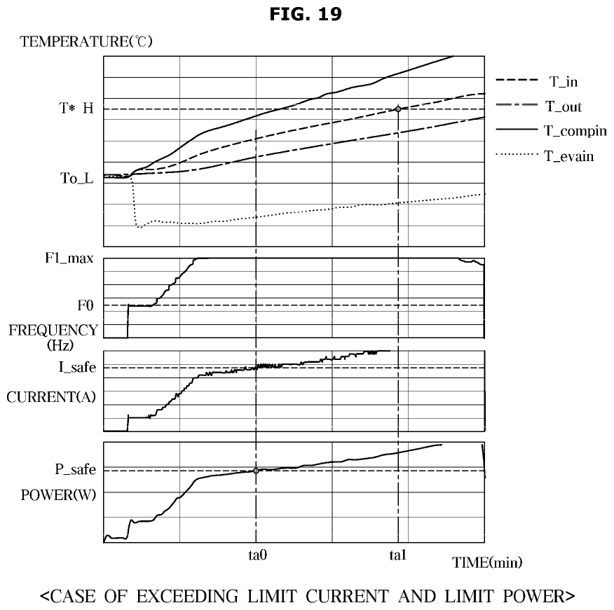

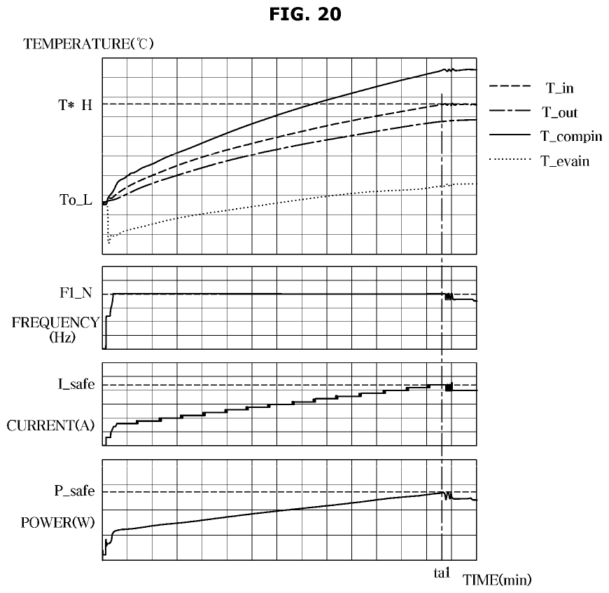

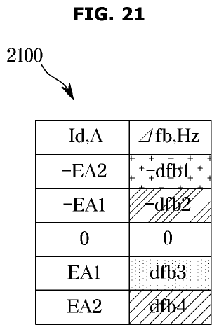

FIG. 18 is a flowchart describing compressor current control in a method of controlling a shoe care apparatus. FIGS. 19 and 20 are graphs illustrating an example in which compressor current control is not applied in a heatup process. FIG. 21 illustrates a current control table.

Referring to FIG. 18, step 1002 is equivalent to what is described above. That is, the compressor 41 may increase the temperature in the chamber 30 by operating at the operation frequency F1 determined based on the target temperature T* and the outside air temperature To, in 1002. In this case, the compressor 41 may operate at the maximum frequency F1_max to reduce heatup time.

Referring to FIG. 19, when the high target temperature T*_H is set in an environment of a low outside air temperature To_L, the compressor 41 may operate at the maximum frequency F1_max to quickly increase the temperature T_in in the chamber 30. In this case, the current applied to the compressor 41 may reach a limit current I_safe before the temperature T_in in the chamber 30 reaches the target temperature T*_H. In other words, as shown in FIG. 19, a point of time ta0 at which the compressor current reaches the limit current I_safe may be earlier than the point of time ta1 at which the temperature T_in in the chamber 30 reaches the target temperature T*_H.

The limit current I_safe may be defined as a maximum current that may operate the compressor 41 without damaging the compressor 41. When the compressor current exceeds the limit current I_safe, a control circuit for controlling the compressor 41 may be damaged. Hence, to prevent damage to the control circuit due to application of an excessive current to the compressor 41, the controller 200 may perform the compressor current control.

In the meantime, an increase in the compressor current has the same meaning as an increase in instantaneous power comsumed by the compressor 41. In other words, even when the instantaneous power of the compressor 41 exceeds limit power P_safe, the control circuit of the compressor 41 may be damaged.

Referring to FIG. 20, when the high target temperature T*_H is set in an environment of the low outside air temperature To_L, the current applied to the compressor 41 may be prevented from reaching the limit current I_safe before the temperature T_in in the chamber 30 reaches the target temperature T*_H by operating the compressor 41 at a normal frequency F1_N. However, there is a downside that it takes a longer time for the temperature T_in in the chamber 30 to reach the target temperature T*_H. Hence, a control method that may efficiently use the maximum frequency of the compressor without damaging the control circuit due to an excessive current is required.

Referring back to FIG. 18, the controller 200 may control the operation frequency of the compressor 41 for the compressor current to be equal to or less than a preset limit current. The controller 200 may control the current applied to the compressor 41 by periodically controlling the operation frequency of the compressor 41. For this, the controller 200 may check whether compressor current I_comp reaches the limit current I_safe, in 1801.

The controller 200 may calculate a current difference Id between the limit current I_safe and the compressor current I_comp at preset intervals, in 1802. The controller 200 may determine a control value Δfb of the operation frequency F1 by using a current control table 2100 stored in advance in 1803, and control the operation frequency based on the control value Δfb in 1804. Controlling of the operation frequency F1 to be applied to the compressor 41 may be performed by adding the control value Δfb to an operation frequency F1(n-1).

The controller 200 may determine the control value Δfb corresponding to the current difference Id by referring to the current control table 2100. The control value Δfb as mentioned in the current control may be referred to as a second control value. For example, in FIG. 21, when the current difference Id is EA1, the control value Δfb may be determined as dfb3. In other words, as the limit current I_safe is larger than the current compressor current I_comp by EA1, the compressor current may be increased to a larger value. When the operation frequency F1 of the compressor 41 is set to be higher by dfb3, the current applied to the compressor 41 increases.

When the first temperature T_in reaches the first threshold temperature T*−λ, the aforementioned fuzzy control may be started in 1003 and 1004.

With this current control, the maximum frequency of the compressor may be efficiently used without damaging the control circuit due to an excessive current. Hence, the heatup time of the air supplied into the chamber may be reduced.

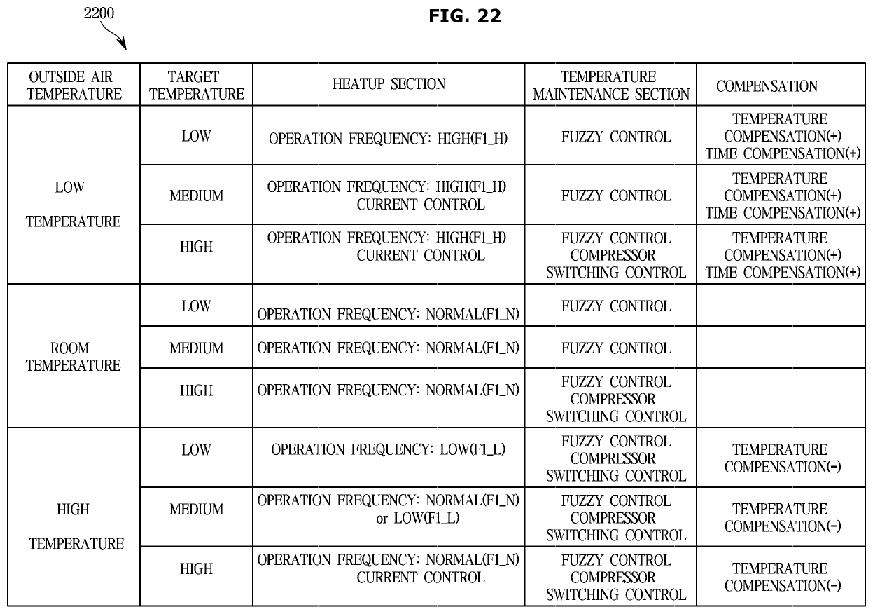

FIG. 22 is a table illustrating embodiments in which the operation frequency of a compressor is controlled based on an outside air temperature and a target temperature.

Referring to table 2200 of FIG. 22, the outside air temperature may be divided into multiple sections. For example, the outside air temperature may be divided into low temperature, room temperature and high temperature. The low temperature may be lower than 15□, the room temperature may be 15□ or higher and 25□ or lower, and the high temperature may exceed 25□. Alternatively, the outside air temperature may be subdivided into more sections.

The target temperature may be divided into multiple sections as well. For example, the target temperature may be divided into low target temperature, medium target temperature and high target temperature. The low target temperature may be 30□ or higher and 38□ or lower, the medium target temperature may be 38□ or higher and 43□ or lower, and the high target temperature may be 43□ or higher and 60□ or lower. Alternatively, the target temperature may be subdivided into more sections.

As the target temperature needs to be maintained regardless of changes in the outside air temperature and the target temperature, all the embodiments may include fuzzy control in common for maintaining the temperature in the chamber 30 at the target temperature.

In an environment having a low outside air temperature, the operation frequency F1 of the compressor 41 may be set to a high value F1_H. The high value F1_H may refer to the maximum frequency F1_max of the compressor 41. This is to reduce the heatup time. When the outside air temperature is low and the target temperature is set to a medium value or higher, current control for controlling the operation frequency of the compressor 41 may be performed. It is because the compressor current may reach the limit current before the temperature in the chamber 30 reaches the target temperature.

In an environment in which the outside air temperature is the room temperature, the operation frequency F1 of the compressor 41 may be set to a normal value F1_N.

When the target temperature is set to have a high value, the outside air temperature is high, or the outside air temperature is high and the target temperature has a high value, compressor switching control may be performed to prevent the temperature in the chamber 30 from exceeding the target temperature or protect the compressor 41.

When the difference between the target temperature and the outside air temperature is small (e.g., when the difference between the target temperature and the outside air temperature is 10□ or less), the operation frequency of the compressor 41 may be set to a low value F1_L. It is to prevent the temperature in the chamber 30 from exceeding the target temperature in the heatup section, In the table 2200 of FIG. 22, illustrated is the operation frequency of the compressor 41 set to the low value F1_L when the outside air temperature is high and the target temperature is set to a low value or a medium value.

When the outside air temperature is high and the target temperature has a high value, the operation frequency of the compressor 41 may be determined as the normal value F1_N.

Furthermore, as there may be a difference between the temperature of the shoes and the outside air temperature, compensation of the target temperature and compensation of the operation time may be performed. For example, in an environment having a low outside air temperature, compensation of the target temperature and compensation of the operation time may be performed positively. It is because the environment having the low outside air temperature requires more heatup time. On the other hand, in an environment having a high outside air temperature, compensation of the target temperature and compensation of the operation time may be performed negatively. As such, more precise temperature control may be implemented by performing compensation of the target temperature and compensation of the operation time.

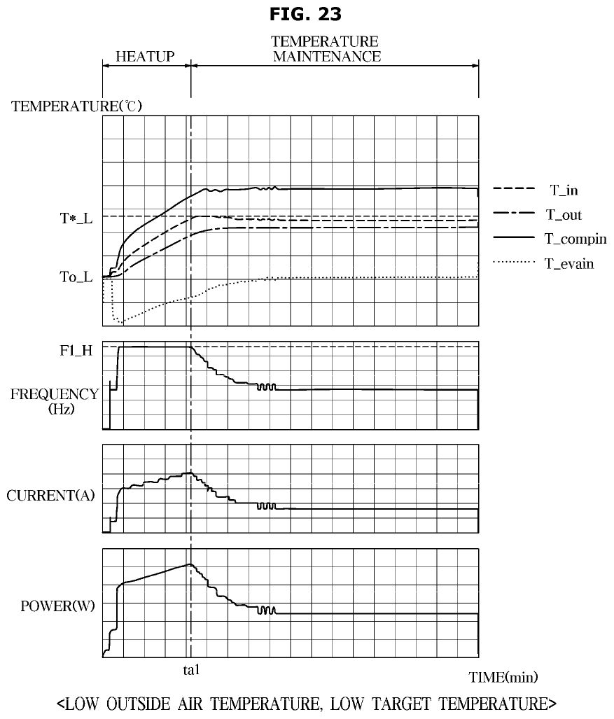

FIGS. 23, 24 and 25 are graphs illustrating control results of a shoe care apparatus according to a target temperature in an environment where outside air temperature is low.

Referring to FIG. 23, when the outside air temperature is low To_L and the target temperature is set to a low value T*_L, the operation frequency of the compressor 41 is set to a high value F1_H. When the temperature T_in of the air flowing into the chamber 30 reaches a first threshold temperature lower than the target temperature T*_L, the controller 200 of the shoe care apparatus 1 performs fuzzy control to maintain the temperature in the chamber 30.

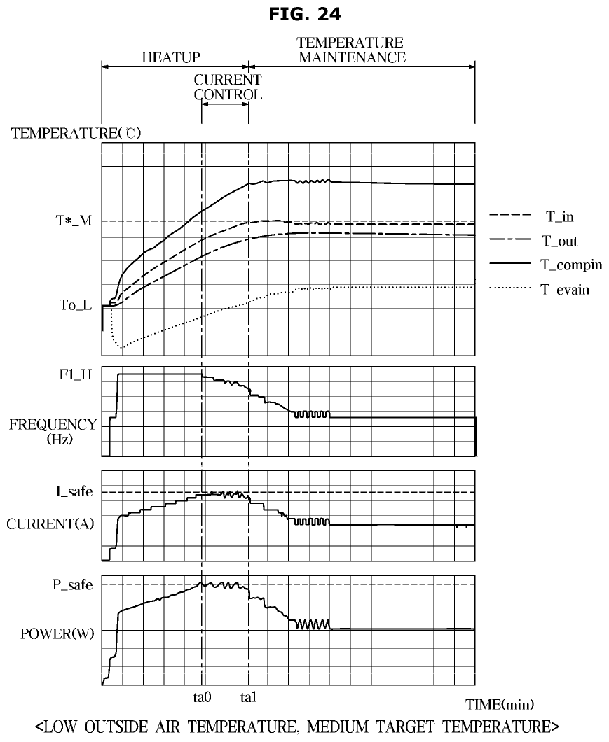

Referring to FIG. 24, when the outside air temperature is low To_L and the target temperature is set to a medium value T*_M, the operation frequency of the compressor 41 is set to a high value F1_H. Furthermore, as the compressor current reaches the limit current I_safe at ta0 before the temperature T_in in the chamber 30 reaches the target temperature T*_H at ta1, current control of the compressor 41 is performed. When the temperature T_in of the air flowing into the chamber 30 reaches the first threshold temperature lower than the target temperature T*_M, the controller 200 of the shoe care apparatus 1 performs fuzzy control to maintain the temperature in the chamber 30.

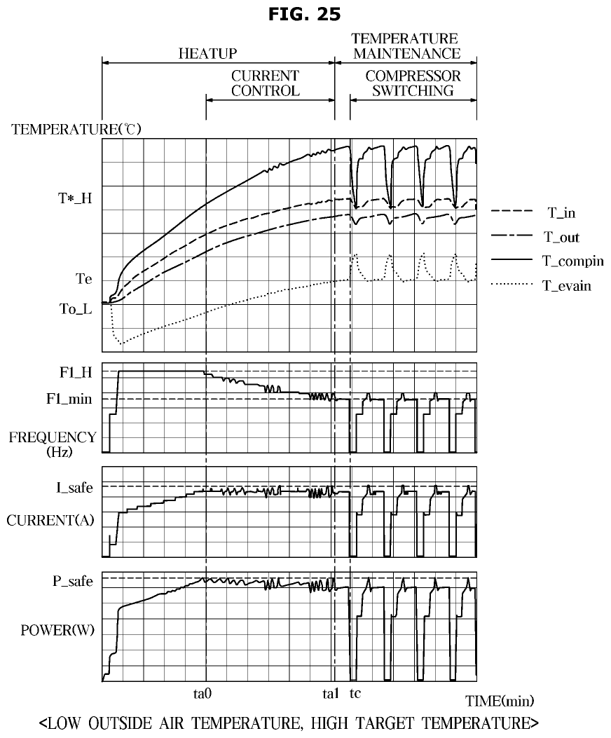

Referring to FIG. 25, when the outside air temperature is low To_L and the target temperature is set to a high value T*_H, the operation frequency of the compressor 41 is set to a high value F1_H. Furthermore, as the compressor current reaches the limit current I_safe at ta0 before the temperature T_in in the chamber 30 reaches the target temperature T*_H at ta1, current control of the compressor 41 is performed. When the temperature T_in of the air flowing into the chamber 30 reaches the first threshold temperature lower than the target temperature T*_H, the controller 200 of the shoe care apparatus 1 performs fuzzy control to maintain the temperature in the chamber 30. Moreover, the controller 200 performs compressor switching control from a point of time tc at which the temperature T_in in the chamber 30 reaches a second threshold temperature T*_H+δ and the operation frequency F1 of the compressor 41 reaches the minimum frequency F1_min. In the meantime, “tc” may be a point of time at which the evaporator inlet temperature T_evain reaches the protection temperature Te.

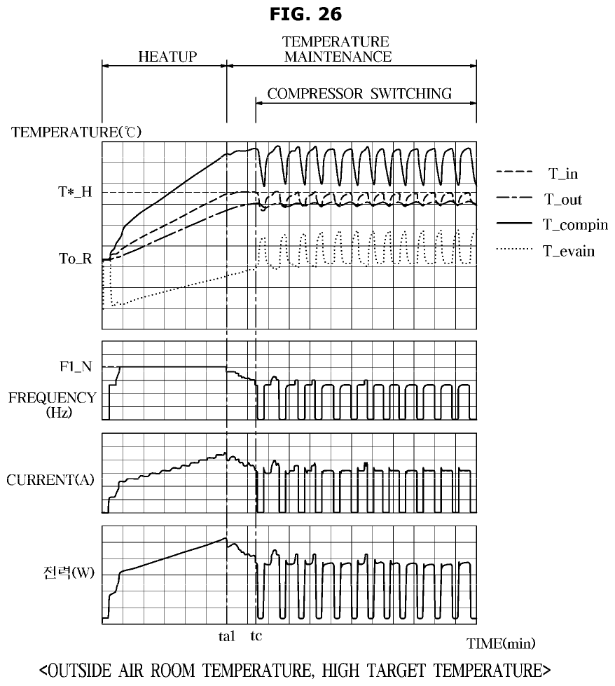

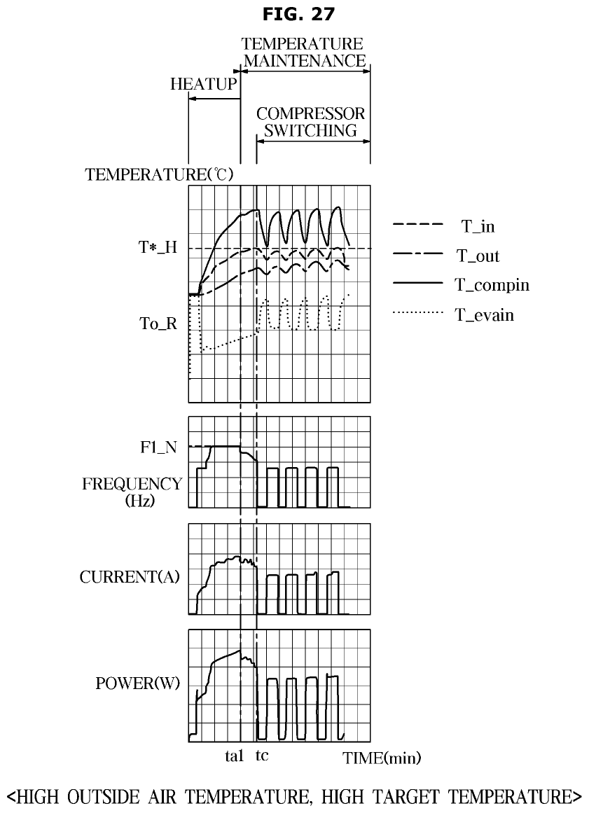

FIGS. 26 and 27 are graphs illustrating control results of a shoe care apparatus according to settings of high target temperature.

Referring to FIG. 26, when the outside air temperature is a room temperature To_R and the target temperature is set to a high value T*_H, the operation frequency of the compressor 41 is set to a normal value F1_N. When the temperature T_in of the air flowing into the chamber 30 reaches the first threshold temperature lower than the target temperature T*_H, the controller 200 of the shoe care apparatus 1 performs fuzzy control to maintain the temperature in the chamber 30. Furthermore, the controller 200 performs compressor switching control from the point of time tc at which the evaporator inlet temperature T_evain reaches the protection temperature Te. In the meantime, “tc” may be a point of time at which the temperature T_in in the chamber 30 reaches the second threshold temperature T*_H+δ and the operation frequency F1 of the compressor 41 reaches the minimum frequency F1_min.

Referring to FIG. 27, when the outside air temperature is low To_L and the target temperature is set to a high value T*_H, the operation frequency of the compressor 41 is set to a high value F1_H. When the temperature T_in of the air flowing into the chamber 30 reaches the first threshold temperature lower than the target temperature T*_H, the controller 200 of the shoe care apparatus 1 performs fuzzy control to maintain the temperature in the chamber 30. Furthermore, the controller 200 performs compressor switching control from the point of time tc at which the evaporator inlet temperature T_evain reaches the protection temperature Te. In the meantime, “tc” may be a point of time at which the temperature T_in in the chamber 30 reaches the second threshold temperature T*_H+δ and the operation frequency F1 of the compressor 41 reaches the minimum frequency F1_min.

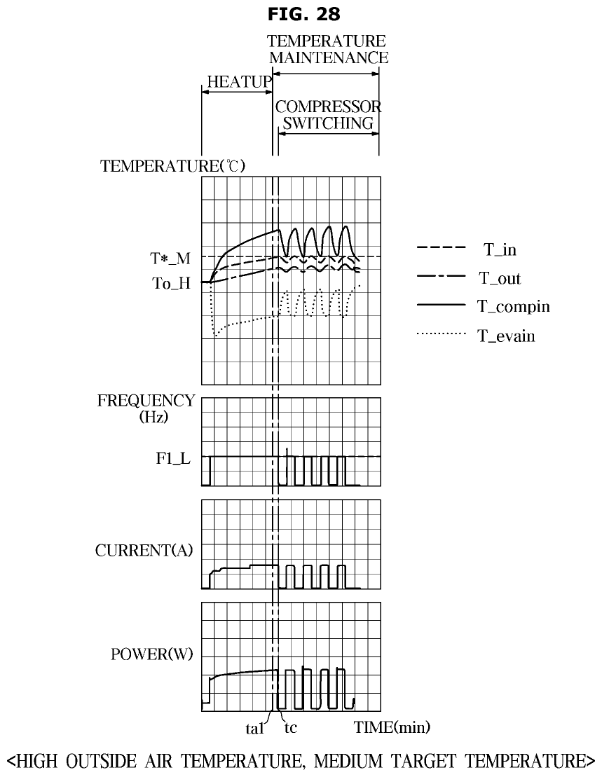

FIG. 28 is a graph illustrating an example in which an operation frequency of a compressor is low.

Referring to FIG. 28, when the outside air temperature is high To_H and the target temperature is set to a medium value T*_M, the operation frequency of the compressor 41 is set to a low value F1_L. In this case, a difference between the outside air temperature and the target temperature may be equal to or less than 100. When the temperature T_in of the air flowing into the chamber 30 reaches the first threshold temperature lower than the target temperature T*_M, the controller 200 of the shoe care apparatus 1 performs fuzzy control to maintain the temperature in the chamber 30. Furthermore, the controller 200 performs compressor switching control from the point of time tc at which the evaporator inlet temperature T_evain reaches the protection temperature Te. In the meantime, “tc” may be a point of time at which the temperature T_in in the chamber 30 reaches the second threshold temperature T*_M+δ and the operation frequency F1 of the compressor 41 reaches the minimum frequency F1_min.

As described above, the shoe care apparatus and method for controlling the same as disclosed may keep the air in a chamber at a constant temperature by circulating the air in the closed apparatus using a heat pump cycle. Accordingly, thermal damage to an object to be dried due to heat buildup in the chamber may be prevented.

Furthermore, the shoe care apparatus and method for controlling the same may reduce the heatup time of air in the chamber by efficiently using a maximum frequency of a compressor without damage to a control circuit due to overcurrent.

According to the disclosure, the shoe care apparatus and method for controlling the same may also keep the environment around the apparatus pleasant because contaminated air produced in the process of drying and deodorizing an abject to be dried is not discharged.

Meanwhile, the embodiments of the disclosure may be implemented in the form of a storage medium for storing instructions to be carried out by a computer. The instructions may be stored in the form of program codes, and when executed by a processor, may generate program modules to perform operation in the embodiments of the disclosure.

The machine-readable storage medium may be provided in the form of a non-transitory storage medium. The term ‘non-transitory storage medium’ may mean a tangible device without including a signal, e.g., electromagnetic waves, and may not distinguish between storing data in the storage medium semi-permanently and temporarily. For example, the non-transitory storage medium may include a buffer that temporarily stores data.

The aforementioned methods according to the various embodiments of the disclosure may be provided in a computer program product. The computer program product may be a commercial product that may be traded between a seller and a buyer. The computer program product may be distributed in the form of a storage medium (e.g., a compact disc read only memory (CD-ROM)), through an application store (e.g., play store™), directly between two user devices (e.g., smart phones), or online (e.g., downloaded or uploaded). In the case of online distribution, at least part of the computer program product (e.g., a downloadable app) may be at least temporarily stored or arbitrarily created in a storage medium that may be readable to a device such as a server of the manufacturer, a server of the application store, or a relay server.

The embodiments of the disclosure have thus far been described with reference to accompanying drawings. It will be obvious to those of ordinary skill in the art that the disclosure may be practiced in other forms than the embodiments of the disclosure as described above without changing the technical idea or essential features of the disclosure. The above embodiments of the disclosure are only by way of example, and should not be construed in a limited sense.

What is claimed is:

1. A shoe care apparatus comprising:
a main body;
a chamber formed in the main body, the chamber including an air inlet and an air outlet;
a first duct connected to the air outlet, the first duct having an evaporator and a condenser arranged inside;
a second duct connected to the first duct and the air inlet;
a holder arranged in the chamber to hold shoes to be cleaned, the holder connected to the air inlet;
a fan configured to circulate air through the first duct, the second duct, the holder, the chamber, and return to the first duct;
a compressor configured to discharge a refrigerant to the condenser;
a first temperature sensor configured to measure a first temperature of air heated by the condenser;
a control panel configured to obtain a user input; and
a controller configured to determine a target temperature of the heated air based on the user input, determine an operation frequency based on the target temperature and an outside air temperature, operate the compressor at the operation frequency, and control the operation frequency of the compressor based on the target temperature and the first temperature.

2. The shoe care apparatus of claim 1, wherein the controller is further configured to
increase or decrease the operation frequency of the compressor for the first temperature to reach the target temperature in response to the first temperature reaching a preset first threshold temperature.

3. The shoe care apparatus of claim 2, wherein the controller is further configured to determine a temperature difference between the target temperature and the first temperature, determine a value of variation in the temperature difference at preset intervals, and determine a control value of the operation frequency corresponding to the temperature difference and the value of variation in the temperature difference by referring to a fuzzy table.

4. The shoe care apparatus of claim 2, wherein the controller is further configured to
stop an operation of the compressor in response to the first temperature reaching a preset second threshold temperature and the operation frequency reaching a preset minimum frequency, and
operate the compressor which has been stopped in response to the first temperature reduced below a preset third threshold temperature.

5. The shoe care apparatus of claim 2, further comprising: a third temperature sensor arranged on an inlet side of the evaporator and configured to measure temperature of the refrigerant,
wherein the controller is further configured to
stop an operation of the compressor in response to the temperature of the refrigerant equal to or higher than a preset protection temperature, and
operate the compressor which has been stopped in response to the first temperature reduced below a preset third threshold temperature.

6. The shoe care apparatus of claim 1, further comprising: a current sensor configured to measure a compressor current,
wherein the controller is further configured to control the operation frequency of the compressor so that the compressor current is equal to or less than a preset limit current.

7. The shoe care apparatus of claim 6, wherein the controller is configured to determine a current difference between the limit current and the compressor current at preset intervals, and determine a control value of the operation frequency corresponding to the current difference by referring to a current control table.

8. The shoe care apparatus of claim 1, further comprising: a second temperature sensor configured to measure a second temperature of air having passed the air outlet,
wherein the controller is further configured to determine the outside air temperature based on the second temperature measured at a beginning of operation of the shoe care apparatus.

9. The shoe care apparatus of claim 1, further comprising: a sterilizer arranged in the chamber or the first duct and configured to sterilize air,
wherein the controller is further configured to
operate the fan and the sterilizer for a preset stabilization time before the operation of the compressor, and
operate the fan and the sterilizer for a preset cooling time after a completion of a dry course in which the compressor is operated.

10. The shoe care apparatus of claim 1, wherein the controller is further configured to determine the target temperature based on a selection of a shoe type and a caring course input through the control panel.

11. A method of controlling a shoe care apparatus including a first duct connected to an air outlet of a chamber, a second duct connected to an air inlet of the chamber, and a holder arranged in the chamber, the method comprising:
determining a target temperature of air to be introduced into the chamber based on a user input obtained through a control panel;
determining an operation frequency of a compressor based on the target temperature and an outside air temperature;
operating the compressor at the operation frequency;
measuring a first temperature of air heated by a condenser arranged in the first duct; and
controlling the operation frequency of the compressor based on the target temperature and the first temperature.

12. The method of claim 11, wherein the controlling of the operation frequency comprises increasing or decreasing the operation frequency of the compressor for the first temperature to reach the target temperature in response to the first temperature reaching a preset first threshold temperature.

13. The method of claim 12, wherein the controlling of the operation frequency comprises
determining a temperature difference between the target temperature and the first temperature;
determining a value of variation in the temperature difference at preset intervals; and
determining a control value of the operation frequency corresponding to the temperature difference and the value of variation in the temperature difference by referring to a fuzzy table.

14. The method of claim 12, further comprising:
stopping an operation of the compressor in response to the first temperature reaching a preset second threshold temperature and the operation frequency reaching a preset minimum frequency; and
operating the compressor which has been stopped in response to the first temperature reduced below a preset third threshold temperature.

15. The method of claim 12, further comprising:
stopping the operation of the compressor in response to a temperature of a refrigerant measured at an inlet of an evaporator equal to or greater than a preset protection temperature; and
operating the compressor which has been stopped in response to the first temperature reduced below a preset third threshold temperature.

16. The method of claim 12, further comprising include measuring a compressor current by a current sensor, wherein the controlling of the operation frequency further includes controlling the operation frequency of the compressor for the compressor current to be equal to or less than a preset limit current.

17. The method of claim 16, where in the controlling of the operation frequency includes determining a current difference between the limit current and the compressor current at preset intervals; and determining a control value of the operation frequency corresponding to the current difference by referring to a current control table.

18. The method of claim 12, further comprising measuring a second temperature of air having passed the air outlet; and determining the outside air temperature based on the second temperature measured at a beginning of operation of the shoe care apparatus.

19. The method of claim 12, further comprising operating a fan and a sterilizer for a preset stabilization time before an operation of the compressor; and operating the fan and the sterilizer for a preset cooling time after a completion of a dry course in which the compressor is operated.

20. The method of claim 12, wherein the determining of the target temperature of the air is based on selecting of a shoe type and a caring course input through the control panel.
